(19) United States
(12) Patent Application Publication
(10) Pub. No.: US 2012/0121526 A1
(43) Pub. Date: May 17, 2012

(54) Composition and method for long-lasting non-permanent straightening of human hair

(76) Inventors: Karen A. Hohenstein, West Hills, CA (US); Serina T. Tran, Northridge, CA (US); Bruce N. Albert, Culver City, CA (US)

(21) Appl. No.: 12/927,385

(22) Filed: Nov. 12, 2010

(51) Int. Cl. A61K 8/44; A61Q 5/00; A61K 8/49

(52) U.S. Cl. 424/70.1

(57) ABSTRACT

A mild reducing composition and method for straightening human hair are described. The composition contains cysteamine hydrochloride in the range of 5-10% by weight at a pH range of 7.0-9.0. The composition is applied to hair and processed for 15-45 minutes. The hair is rinsed with water, dried with a blow-dryer and ironed with a straightening iron. The hair is allowed to air-oxidize for 24-72 hours. The resultant straightening lasts 2 to 4 months, and the hair gradually reverts to the pre-treatment state with no obvious re-growth. The treatment may be re-applied every 2 to 4 months without significant damage.

### BACKGROUND OF THE INVENTION

(1) Field of the Invention

The present invention relates to a cosmetic reducing composition containing cysteamine hydrochloride, and its use in a process for straightening human hair.

(2) Description of the Related Art

Methods for straightening human hair include the use of relaxer creams containing “lye” (sodium hydroxide or potassium hydroxide), or the newer “no-lye” relaxer creams containing calcium hydroxide or lithium hydroxide, mixed with a liquid containing guanidine carbonate. These products cleave the disulfide bonds in hair, thereby softening it. The hair is manipulated into a straight configuration by the operator during the processing time. After the hair is sufficiently processed, the hair is rinsed, and a low pH “neutralizing shampoo” is applied to form new sulfur bonds that maintain the hair in the desired straight configuration. With these systems, the disulfide bond in cystine, an amino acid contained in the hair keratin protein, is cleaved and a new sulfide bond is formed, creating the amino acid. lanthionine, with one sulfur atom. The second sulfur atom is eliminated in the neutralizing step as hydrogen sulfide. Hair treated with these systems is very straight, and much weaker than untreated hair. The pH of the products is in the very high range of pH 10-12, a level that is corrosive to skin and eye. Users frequently experience stinging of the scalp, and may suffer skin burns or eye damage. Breakage of the hair is very common, and the hair is left dull and limp. As the chemistry of the hair is fundamentally altered by this process, relaxed hair cannot be treated with “thio”-type straightening products.

Another method for straightening hair utilizes traditional permanent wave chemistry with modifications in the technique. These products are based on mercaptans or their esters, “thio” compounds such as ammonium thioglycolate, ethanolamine thioglycolate, glyceryl monothioglycolate or thioglycolic acid. An alkali, such as ammonium hydroxide, an alkanolamine, or an amino alcohol, is typically added to the system to swell the hair and enhance penetration of the reducing agent. The pH values for these systems are typically within the range 7.5-9.5. In this method, the “thio” reducing mixture is applied to hair, allowed to process for 15-45 minutes while the operator smoothes the hair. The hair is rinsed, and the oxidant, generally a solution of hydrogen peroxide or sodium or potassium bromate, is applied to reform the disulfide bonds in the new straight configuration. The hair is rinsed, then dried with a blow dryer and usually ironed with a straightening iron. In the so-called “Japanese” straightening or “thermal” straightening process, after the reducing mixture is rinsed, the hair is dried with a blow dryer, and ironed with a straightening iron. The oxidant is then applied to the hair, allowed to process for 5-15 minutes, and rinsed. This modification produces better straightening, although even more hair damage. Hair treated with “thio” products is generally brittle and more easily damaged by styling tools such as blow dryers and straightening irons.

Straighteners based on cysteine and cysteamine salts are fairly new to the market. The chemistry is similar to the “thio” straighteners. As with the “thio” systems, the cysteine or cysteamine reducing mixture is applied to hair, and allowed to process for 15-45 minutes while the operator smoothes the hair. The hair is rinsed, the oxidant solution is applied and allowed to process for 5-15 minutes, then rinsed again. The hair is dried in a straight configuration with a blow dryer, and ironed with a straightening iron. Compared to the “thio” systems, cysteine and cysteamine are gentler on the hair, cause less damage, but are also less effective straighteners. In addition, more “reversion,” or return to the hair's original curly configuration with subsequent shampooing, is noted with cysteine and cysteamine systems.

The newest and most controversial method for hair straightening is the so-called “Brazilian” straightener, based on formaldehyde. In this method, a solution containing approximately 2-15% formaldehyde is applied to the hair, the hair is dried with a blow dryer, then ironed with a very hot straightening iron at approximately 400-450 deg F. (200-235 deg C.). The client must not re-wet the hair for 48-72 hours so that the salt bonds in the hair harden in the new straight configuration. The client must use special shampoos and conditioners that are free of Sodium Chloride to maintain the straight configuration. As this system does not affect the disulfide bonds, the straightening is not permanent, and the hair will gradually return to its original configuration in approximately two to four months. Although this method is quite popular in salons, Formaldehyde is a hazardous chemical, listed as a known carcinogen under California Proposition 65. The vapors produced by the formaldehyde during the drying process and especially during the ironing process are hazardous to both client and operator, as well as other employees and patrons present in the salon. In addition, most of the “Brazilian” products do not disclose ingredients, so that users are unaware of their exposure to harmful chemicals.

Development of a is a product that can provide long-lasting yet non-permanent straightening to the hair and will revert to the pre-treatment state with normal shampooing over a period of two to four months, with no obvious line of demarcation of untreated new hair growth, and with minimal damage to the structure of the hair represents a great improvement in the field of hair treatment and satisfies a long felt need of the hairdresser and client.

### SUMMARY OF THE INVENTION

The present invention is a product that provides long-lasting yet non-permanent straightening to the hair that will revert or return to the pre-treatment state with normal shampooing over a period of two to four months, with no obvious line of demarcation of untreated new hair growth, and with minimal damage to the structure of the hair.

The present invention is a composition capable of reducing disulfide bonds in the keratin protein of human hair. It comprises:

cysteamine hydrochloride in the range of 5-10% by weight;

an amino acid in the range of 0.01-10% by weight;

sufficient alkali to produce a pH in the range of 7.0-9.0.

The alkali is preferably ammonium hydroxide, an alkali metal hydroxide, an alkanolamine, an amino alcohol, or an amino acid.

The amino acid is preferably lysine, histidine or arginine.

The preferred alkali metal hydroxide is sodium hydroxide

The alkanolamine is preferably monoethanolamine or triethanolamine.

The preferred amino alcohol is aminomethyl propanol

This composition is applied to human hair for 15-45 minutes. This reduces disulfide bonds, softens the structure of the hair and allows it to be reconfigured. The equation for this reaction is:

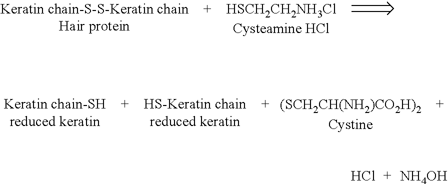

Thereafter, the hair is rinsed with water to remove the compositions, dried in a straight configuration with hot air and ironed with a straightening iron. The hair becomes oxidized by exposure to atmospheric oxygen for a period of 24 to 72 hours during which period said hair must not be wetted. The equation for the oxidization reaction is:

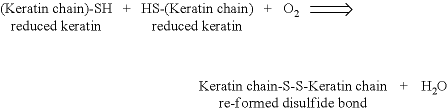

An appreciation of the other aims and objectives of the present invention and an understanding of it may be achieved by referring to the accompanying drawings and description of a preferred embodiment.

### DETAILED DESCRIPTION OF THE PREFERRED EMBODIMENT

While the present invention is described herein with reference to illustrative embodiments for particular applications, it should be understood that the invention is not limited thereto. Those having ordinary skill in the art and access to the teachings provided herein will recognize additional modifications, applications, and embodiments within the scope thereof and additional fields in which the present invention would be of significant utility.

Generally, the composition contains cysteamine hydrochloride present in an amount of 5% to 10% by weight, particularly about 6% to 8% by weight. An amino acid such as lysine, histidine or arginine is present in an amount of 0.01% to 10% by weight, preferably about 1% to 5% by weight. The pH of the system is adjusted within the range of pH 7.0-9.0, preferably pH 7.5-8.5, with an alkali selected the group consisting of ammonium hydroxide, alkali metal hydroxides, such as sodium hydroxide, alkanolamines such as monoethanolamine or triethanolamine, amino alcohols such as aminomethyl propanol, or basic amino acids, such as lysine, histidine or arginine.

Optionally, thickeners (e.g. hydroxyethylcellulose and xanthan gum), fatty alcohols (e.g. cetyl alcohol, cetearyl alcohol and stearyl alcohol) and emulsifiers (e.g. Ceteareth-20; Polawax available from Croda, Fort Lee, N.J.; and Crodafos CES available from Croda, Fort Lee, N.J.) may be included in the composition to provide a cream or gel consistency. Hair softening and moisturizing agents such as glycerine or triglycerides may be included for additional hair conditioning effect. Sequestrants (e.g. disodium EDTA, tetrasodium EDTA) and preservatives (e.g. sodium hydroxymethylglycinate or Suttocide A, available from ISP, Chatham, N.J.; methylparaben; and phenoxyethanol may be added to enhance the stability of the composition. Fragrance may be added to mask the malodor produced by the process.

The present invention also relates to a method for straightening hair in which a composition as defined above is applied to the hair. The composition is allowed to process for 15-45 minutes, preferably from 20 to 30 minutes. The hair is rinsed with water to remove the composition. The hair is then dried in a straight configuration with a blow dryer, and ironed in a straight configuration with a straightening iron. The straightened hair is allowed to oxidize to re-form the disulfide bonds by exposure to atmospheric oxygen for a period of 24-72 hours before the hair is rewetted. Human hair treated with this method was significantly straighter. The hair was soft and glossy, with no apparent damage. The straightening was durable, lasting approximately 2 to 4 months, and the hair gradually reverted to the pre-treatment curly or wavy state with normal shampooing. No obvious line of demarcation between treated hair and new untreated hair growth was noted.

Optionally, a leave-in treatment or styling product may be applied to the hair prior to drying with the blow dryer or ironing with the straightening iron. Any of the many different hair leave-in treatments and styling products available on the market may be used.

By way of illustration and without any limiting nature, several examples of reducing compositions and methods for their use according to the invention will now be given.

### EXAMPLE 1

Purified water q.s. to 100.00%

Glycerin 99.5% USP 3.00%

Cetearyl alcohol (and) dicetyl phosphate (and) ceteth-10 phosphate (Crodafos CES available from Croda of Edison N.J.) 10.00%

Glycine soya (soybean) oil 8.00%

Purified water 10.00%

Cysteamine hydrochloride (Oristar CAHC available from Orient Stars, Gardena Calif.) 6.00%

Arginine USP (available from Ajinomoto, Fort Lee N.J.) 1.00%

Aminomethyl propanol 95% (AMP Ultra PC 2000 available from Dow, Midland Mich.) 2.00%

The mixture was a soft cream, pH 8.00. The mixture was applied to curly human hair and allowed to process for 20 minutes. The hair was rinsed with warm water, then dried straight with a blow dryer and ironed with a straightening iron. The hair was allowed to air-oxidize for a period of 24 hours. The hair was then shampooed and allowed to air-dry. The new configuration of the hair was significantly straighter, with a soft, open wave pattern.

### EXAMPLE 2

Purified water q.s. to 100.00%

Glycerin 99.5% USP 3.00%

Cetearyl alcohol (and) dicetyl phosphate (and) ceteth-10 phosphate (Crodafos CES available from Croda, Edison, N.J.) 10.00%

Glycine soya (soybean) oil 10.00%

Purified water 10.00%

Cysteamine hydrochloride (Oristar CAHC available from Orient Stars, Gardena Calif.) 6.00%

Arginine USP (available from Ajinomoto, Fort Lee N.J.) 4.00%

The mixture was a soft cream, pH 7.92. The mixture was applied to curly human hair and allowed to process for 30 minutes. The hair was rinsed with warm water, then dried straight with a blow dryer and ironed with a straightening iron. The hair was allowed to air-oxidize for a period of 48 hours. The hair was then shampooed and allowed to air-dry. The new configuration of the hair was almost straight.

### EXAMPLE 3

Purified water q.s. to 100.00%

Disodium EDTA 0.2%

Glycerin 99.5% USP 3.00%

Cetearyl alcohol (and) dicetyl phosphate (and) ceteth-10 phosphate (Crodafos CES available from Croda, Edison N.J.) 10.00%

Glycine soja (Soybean) Oil 10.00%

Oleth-5 phosphate (and) dioleyl phosphate (Crodafos HCE available from Croda, Edison N.J.) 1.00%

Purified water 10.00%

Cysteamine hydrochloride (Oristar CAHC available from Orient Stars, Gardena Calif.) 7.50%

Arginine USP (available from Ajinomoto, Fort Lee N.J.) 5.00%

The mixture was a soft cream, pH 7.94. The mixture was applied to curly human hair and allowed to process for 30 minutes. The hair was rinsed with warm water, then dried straight with a blow dryer and ironed with a straightening iron. The hair was allowed to air-oxidize for a period of 48 hours. The hair was then shampooed and allowed to air-dry. The new configuration of the hair was essentially straight.

Thus, the present invention has been described herein with reference to a particular embodiment for a particular application. Those having ordinary skill in the art and access to the present teachings will recognize additional modifications, applications and embodiments within the scope thereof

It is therefore intended by the appended claims to cover any and all such applications, modifications and embodiments within the scope of the present invention.

What is claimed is:

1. A composition capable of reducing disulfide bonds in the keratin protein of human hair comprising:
(a) cysteamine hydrochloride in the range of 5-10% by weight;
(b) an amino acid in the range of 0.01-10% by weight;
(c) sufficient alkali to produce a pH in the range of 7.0-9.0.

2. A composition as claimed in claim 1 in which said alkali is selected from the group consisting of ammonium hydroxide, an alkali metal hydroxide, an alkanolamine, an amino alcohol, and an amino acid.

3. A composition as claimed in claim 1 or 2 in which said amino acid is selected from the group consisting of lysine, histidine and arginine.

4. A composition as claimed in claim 2 in which said alkali metal hydroxide is sodium hydroxide

5. A composition as claimed in claim 2 in which said alkanolamine is selected from the group consisting of monoethanolamine and triethanolamine.

6. A composition as claimed in claim 2 in which said amino alcohol is aminomethyl propanol

7. A method of straightening human hair comprising the steps of:
(a) applying the composition of claim 1 to the hair for 15-45 minutes, thereby reducing disulfide bonds, softening the structure of the hair and allowing it to be reconfigured;
(b) rinsing said hair with water to remove the composition of claim 1;
(c) drying the hair in a straight configuration with hot air;
(d) ironing said hair with a straightening iron;
(e) allowing said hair to become oxidized by exposure to atmospheric oxygen for a period of 24 to 72 hours during which period said hair must not be wetted.
